(19) United States
(12) Patent Application Publication
(10) Pub. No.: US 2020/0065649 A1
(43) Pub. Date: Feb. 27, 2020

(54) Method for robust and noise-tolerant SpO2 determination

(71) Applicants: Odd Inge Sandbekkhaug, Allen, TX (US); Surinder Pal Singh, Mohali (IN); Divesh Sisodia, Kharar (IN)

(72) Inventors: Odd Inge Sandbekkhaug, Allen, TX (US); Surinder Pal Singh, Mohali (IN); Divesh Sisodia, Kharar (IN)

(21) Appl. No.: 16/107,919

(22) Filed: Aug. 21, 2018

(51) Int. Cl. G06N 3/04; G16H 40/63; G06N 3/08

(52) U.S. Cl. CPC G06N 3/0445; G06N 3/08; G16H 40/63

(57) ABSTRACT

A recurrent neural network model is trained to ignore noise components and accurately reconstruct quasi-periodic SpO2 signal waveforms. In accordance with the invention, the neural network is trained on a carefully structured data set so as to be able to (1) be able to use deep learning techniques for model training, and (2) utilize traditional time-series forecasting neural network techniques to produce a clean reconstructed signal from potentially noisy inputs. A novel technique is used to construct a training data set that turns a forward-looking RNN forecasting model into a “sideways-looking” model which acts as a sophisticated noise filter.

### CROSS REFERENCE TO RELATED APPLICATIONS

None.

### TECHNICAL FIELD

This invention relates to pulse oximeter devices which measure the oxygen saturation (SpO2) of hemoglobin and, in particular, to an improved system for accurate signal acquisition and measurement in the presence of ambient noise and interference such as caused by patient motion artifacts.

### BACKGROUND ART

This section contains examples of existing patents related to SpO2 measurement which try to solve similar problems. Related prior art falls into three main categories: Combating noise by physical device modifications, combating noise by improved signal processing algorithms, and combating noise by applying artificial intelligence.

### Combating Noise by Physical Device Modifications

In order to combat noise, approaches have been developed to maintain the measurement probe in a fixed position relative to the patient body, thereby limiting the possibility of noise artifacts due to movement. These inventions somewhat improve the reliability of signal acquisition but do not effectively address noise. Some representative examples of patents in this category include:

U.S. Pat. No. 8,396,527B2 Medical sensor for reducing signal artifacts and technique for using the same

U.S. Pat. No. 8,260,391 B2 Medical sensor for reducing motion artifacts and technique for using the same

U.S. Pat. No. 8,190,224B2 Medical sensor for reducing signal artifacts and technique for using the same

U.S. Pat. No. 7,890,153B2 System and method for mitigating interference in pulse oximetry

U.S. Pat. No. 7,720,516B2 Motion compatible sensor for non-invasive optical blood analysis

### Combating Noise by Improved Signal Processing Algorithms

The most popular methods for dealing with noise is by improved signal processing algorithms. These inventions implement various signal filtering and enhancement methods, but tend to have side-effects such as filtering or otherwise perturbing the base signal. Additionally these hand-engineered signal processing algorithms can be complex to use and can have very narrow effectiveness, so an ensemble of different algorithms may be needed, increasing the complexity of the device. Some representative examples of patents in this category include:

U.S. Pat. No. 6,987,994B1 Pulse oximetry SpO2 determination

U.S. Pat. No. 6,385,471 B1 System for pulse oximetry SpO2 determination

U.S. Pat. No. 7,274,955B2 Parameter compensated pulse oximeter

### Combating Noise by Applying Artificial Intelligence

Several patents exist related to noise reduction using Artificial Intelligence (Al) and Neural Network technology, mostly in the area of audio processing. These methods apply neural networks in narrow role such as an improved filter, chiefly to extract (and boost) particular features from an audio signal. The application of Al as an intelligent filter operates on arbitrary signals, and differs from our proposed invention because we bias our Al to emphasize recognition of a particular set of waveform patterns. Some representative examples of patents in this category include:

U.S. Pat. No. 5,742,694A Noise reduction filter

U.S. Pat. No. 7,082,394B2 Noise-robust feature extraction using multi-layer principal component analysis

U.S. Pat. No. 8,543,526B2 Systems and methods using neural networks to reduce noise in audio signals

US20080037804A1 Neural network filtering techniques for compensating linear and non-linear distortion of an audio transducer

### SUMMARY OF THE INVENTION

### Technical Problem

Pulse oximeters provide an effective way to non-intrusively measure the oxygen saturation (SpO2) of arterial hemoglobin, but the sensors used in these devices are sensitive to noise which in turn results in invalid SpO2 readings.

A pulse oximeter includes a probe that is placed on some appendage which is cutaneous vascular, such as the fingertip. The probe contains two light emitting diodes, each of which emits light at a specific wavelength, one in the red band and one in the infrared band. The amount of light transmitted through the intervening fingertip is sampled many times per second at both wavelengths. Photoplethysmography (PPG) is an optical technique that exploits the wavelength-dependent variation in light absorption coefficients for different tissues to detect blood volume changes in the microvascular bed of tissue. An increase in blood volume will result in an increase in the optical path length, and thus a decrease in the intensity of the transmitted light. The resulting sampled light intensity manifests itself as a PPG signal waveform consisting of a baseline (DC) component and a pulsative (AC) component at the cardiac frequency. The PPG waveform [101] is similar in morphology to the waveform obtained from arterial blood pressure monitors. From this PPG waveform it is possible to compute Heart Rate (HR), blood perfusion and respiration rate.

The oxygen saturation of the hemoglobin in arterial blood is determined by the relative proportions of oxygenated hemoglobin and reduced hemoglobin in the arterial blood. A pulse oximeter calculates the SpO2 by measuring the difference in the absorption spectra of these two forms of hemoglobin.

The data readings from the probe sensor is significantly impacted by the presence of noise, so reducing or eliminating noise artifacts is a key challenge in achieving accurate biometric readings. Any transient change in position of the light emitting diodes relative to the intervening tissue and light detector will introduce errors in the sampled signal. Movement artifacts caused by the patient can mimic vascular beats within the normal physiological range (thereby producing a false but apparently valid signal component), or they could sufficiently distort the measurements to the point where a signal can't be reliably extracted. SpO2 devices based on the current state of the art have difficulty providing a reliable reading in the presence of noise, and either must apply an ensemble of filters or elect to discard noisy signals from processing.

### Solution to the Problem

The above-described problems are solved and a technical advance achieved in the field by the improved system for non invasively calculating the oxygenation of hemoglobin in arterial blood using a pulse oximeter. This improved system takes advantage of advances in the fields of Neural Networks and Artificial Intelligence to ignore the noise component in the acquired SpO2 signals.

Our method utilizes an Artificial Intelligence (Al) neural network trained to recognize the real physiological signal even in the presence of noise. Rather than trying to eliminate noise, the novel approach is to accept the noisy signal and simply allow a carefully trained neural network to ignore the noise components and reconstruct the original signal. We use a special kind of neural networks, Recurrent Neural Networks (RNN), which have all the properties of a traditional neural network but with an added dynamic memory component, allowing the neural network to recognize and construct order-dependent sequences of values. Trained on a carefully curated set of training data, the RNN can accurately discard noise artifacts and deliver a clean measurement which reflects the real physiological signal.

The approach of training and using the RNN to map the whole input signal to an output signal is significantly simpler than training neural networks to act as intelligent filters which then need to be integrated with more complex signal processing algorithms.

A quick summary of Neural Networks

It is not the intention to provide an in-depth explanation of Neural Networks here, but we will highlight the most important aspects which relate to the invention.

Neural networks are trained to recognize patterns in data by consuming examples of known input data (the training set) and an encoding of what the input data means (the label). Each individual entry/observation in the training set is labeled with its encoding, and with sufficient number of examples the neural network is able to learn a generalized model for how input data maps to labels. Once trained, the generalized model can be used to generate meaningful predictions for input data it has never seen before.

As one simple example, one could design a neural network model to convert from Fahrenheit to Celsius by creating a training set [201] with several temperature observations in Fahrenheit and corresponding labels as the Celsius equivalent. With enough training examples, the neural network will learn to convert any number from Fahrenheit to Celsius without ever knowing the mathematical conversion function.

Recurrent Neural Networks (RNN) are a particular kind of neural networks which incorporate a “memory” function which allows it to recognize not only mappings of individual values, but also sequences of values and mappings where the order of individual values matters. RNNs are frequently used in time-series analysis due to their ability to predict future values based on recently observed values.

A common approach is to construct a training set [301] such that the label associated with the input value at time step t is equal to the input value at time step t+1. In this way the RNN is trained to recognize the general shape of the time series, and to predict based on the value at time step t (and based on the sequence of n previously observed values t−n . . . t−1, where n is the number of time steps to look/remember backwards) what the most likely value will be for time step t+1 [302]. This approach is generally known as “time-series forecasting”.

Another way to visualize this can be seen in figure [303]. At time step t we forecast the value at t+1 by looking at the label at time t. It corresponds to the next value in the time series, the signal at time t+1. In order to learn a forecasting model, the label is simply the input data shifted by one time-step into the future.

Note that it is not enough to simply know the sampled value at time=t in order to forecast the value at t+1 since time-series signals can have have both rising and falling slopes, and the next value in the time series depends on which direction the signal is going. This is exactly the problem which the memory component of the RNN solves. Whereas traditional Neural Networks work well with “snapshot” values, RNNs work well with values in the context of trends and momentums.

### Our Approach: Time-Series “Side-Casting”

A conventional SpO2 device uses a probe [401] with basic signal processing [402] to separate the red and infra-red samples. Some additional signal processing is usually applied to filter noise from the signal. Prior art uses hand-engineered signal processing algorithms [403] to achieve this. These signals form a PPG waveform which is then sent to a conventional SpO2 processing unit [404].

Our approach improves on this traditional architecture and utilizes an RNN which is trained to detect PPG waveforms in place of hand-engineered filtering algorithms. The RNN takes as input the signals sensed by the conventional SpO2 probe [501] and conventional basic signal processing to separate the red and infrared components [502]. Our method achieves improved performance by using a pre-trained RNN model [503] to detect the PPG waveform in place of hand-engineered filtering algorithms. Once the PPG waveform has been identified by the RNN, the RNN is capable of ignoring noise in the signal and returns a reconstructed signal which is noise-free and can be passed on to a conventional SpO2 apparatus [504].

The general shape of the PPG waveform is learnt from the training data set, and due to the dynamic memory component of RNN, the neural network is able to quickly tune itself to match the PPG waveform unique to each patient during runtime.

The architecture of the neural network itself and the structure of the training data affects how well the RNN performs. A common approach is to do some level of “feature engineering” on the data before feeding it to the neural network. By feature engineering we mean pre-processing of the data set and selecting a subset of the data upon which to base the neural network on. This approach is similar to traditional hand-engineered signal processing algorithms, which applies neural networks on the hand-engineered features.

Our approach utilizes a technique known as “End-to-end Deep Learning” which allows for training of neural networks on data sets without doing any feature engineering, but with the tradeoff that the neural networks may require more layers and require much more training data. With deep learning, the neural network effectively does the feature engineering on its own. From an overall approach, it is much simpler to apply, and moves a lot of complexity away from algorithmic feature engineering and into the neural network.

Our novel approach is to develop an RNN that maps a noisy signal sample to a clean signal. Instead of predicting the signal (t+1) from signal (t), we train the RNN to predict the true PPG signal (t) [601] from a noisy PPG sample (t) [602] by feeding it a data set which contains noisy signals. This is possible because the pulsative component of the PPG is present in the raw signal even during conditions of noise. We use the clean signal at time t as the label [606] for the corresponding noisy signal at time t. By adding a variety of known noise components [603] to the training set [607], the RNN [604] learns to ignore generalized noise components and reconstruct the base PPG signal [605].

RNNs are traditionally engineered to look at a time series backwards in order to learn what the most likely values will be going forward. The same time series is used for both training data and training labels, just shifted one or more time steps forward. Our approach uses a separate parallel time series as the labels, and therefore can be imagined to look not forward at time-shifted labels, but sideways. [608]

We name this new approach “time-series side-casting” because we are not forecasting (predicting) a future value at time=t+1 based on forward-shifted labels, but rather looking “sideways” for labels at time=t to predict the true value at time=t.

Neural Network model considerations

The neural network architectures of Recurrent Neural Network (RNN) takes advantage of a memory function which allows neural networks to recognize sequences of values (i.e. waveform shapes) and they exhibit dynamic temporal behavior for a time sequence.

Several sub-types of Recurrent Neural Networks exist, and we observe best performance using Long Short Term Memory (LSTM) nodes and in particular several layers of LSTMs (also known as “stacked LSTM” models). Other RNN sub-types may achieve better performance depending on the particular waveform and data set, and our solution is not limited to LSTM implementations but can be applied to a variety of RNN types.

The trained RNN model is represented by a series of model weights which characterize the behavior and patterns learnt from the training data. The model can be instantiated in any programmatic environment, including embedded firmware, software and FPGA.

### Engineering the Training Set

Noise components can be simulated or sampled from real-life signals. Because it is difficult to accurately quantify and isolate noise from real-world signals, we construct a synthetic training set which is based on a wide range of noise frequencies and amplitudes (including samples with zero noise), along with PPG waveforms within possible physiological ranges. By adding a large amount of varied synthetic and real-world noise data we achieve a large enough training set to be of practical use.

By mixing in a wide range of noise signatures into the true PPG signal, and using the true PPG signal as the label for the resulting noisy signal, the RNN learns to distinguish the PPG waveform component from the noise.

The construction of the training data set is crucial, and we ensure that the data set has (1) a variety of valid (true) PPG signals of different frequencies and amplitudes, and (2) a variety of noise patterns of different frequencies and amplitudes, including random noise

Noise is generated by a software simulator capable of creating a virtually infinite data set of noise patterns and valid PPG waveforms. PPG waveforms and noise signatures recorded from a real person can be used as basis for further augmentation of the training set.

### Advantageous Effects of Invention

The key advantage of the invention is that SpO2 devices deliver accurate results in presence of noise, and can therefore be used in a wider range of applications.

The invention can reduce the number of corner-cases which are not covered by traditional signal processing methods. At low perfusion levels, motion artifacts and noise are more prevalent and reduces the effective signal-too-noise ratio. By using our RNN approach, higher levels of noise and interference may be tolerated.

The traditional methods of hand-engineering (fixed) signal processing algorithms are prone to poor performance in corner-cases. Noise generation is a simpler task than noise/motion artifact elimination, and so the RNN approach delivers more robust performance even in corner-cases when the data set is sufficiently large. To improve device performance, we simply add more permutations of training data.

The RNN approach can work on any standard probe and data acquisition apparatus, and is an optional addition to improve on existing signal acquisition.

It is possible that the RNN approach will reduce or eliminate the need for fixed algorithms in noise cancellation and signal extraction.

In cases where the RNN is embedded in FPGA, our approach can lead to further improvements in total hardware cost.

Traditional approaches to combat noise artifacts in pulse oximeters depend on improving the robustness of the probe itself (trying to eliminate probe slippage and wander) or in other ways reduce the the source of the noise, namely patient movement. Since patient-induced artifacts can not always be controlled due to involuntary movements (such as in patients with hypothermia or Parkinson's disease) these approaches have limited application. More robust probes are also more expensive, limiting their use in low-cost applications. Because the new invention can work in the presence of noise, we enable a wider field of application environments and can accept simpler and lower-cost probes.

### BRIEF DESCRIPTION OF DRAWINGS

FIG. 1 is an illustration of a PPG Waveform, showing Red and Infra Red components of the same signal over time.

FIG. 2 is a simplified example of a training set for mapping from Fahrenheit to Celcius

FIG. 3 is a simplified example a training set for leaning a time sequence (waveform)

FIG. 4 is a block diagram of the data flow in a typical pulse oximeter device.

FIG. 5 is a block diagram which shows the location of the RNN in the improved invention.

FIG. 6 is a block diagram which shows the relationship between the training data, label and the RNN itself.

FIG. 7 shows an example of the RNN model embodied as part of the SpO2 sensor device

FIG. 8 shows an example of the RNN model embodied in an external (to the SpO2 sensor) computing device

FIG. 9 shows an example of the RNN model embodied in an external (to the SpO2 sensor) computing environment, such as a cloud-based environment.

FIG. 10 is an example of the RNN model performance, showing for the RED channel the pure signal (label), the noise-added signal, and the reconstructed (predicted) signal.

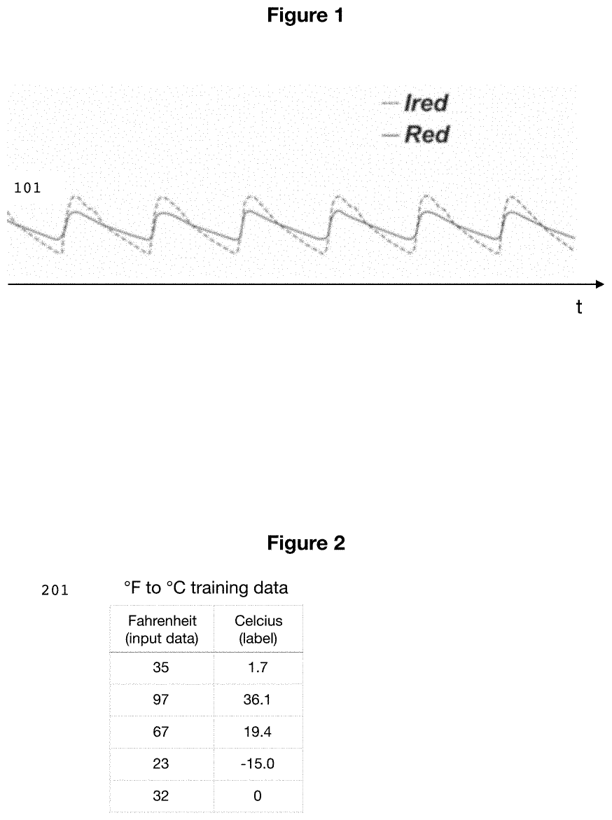

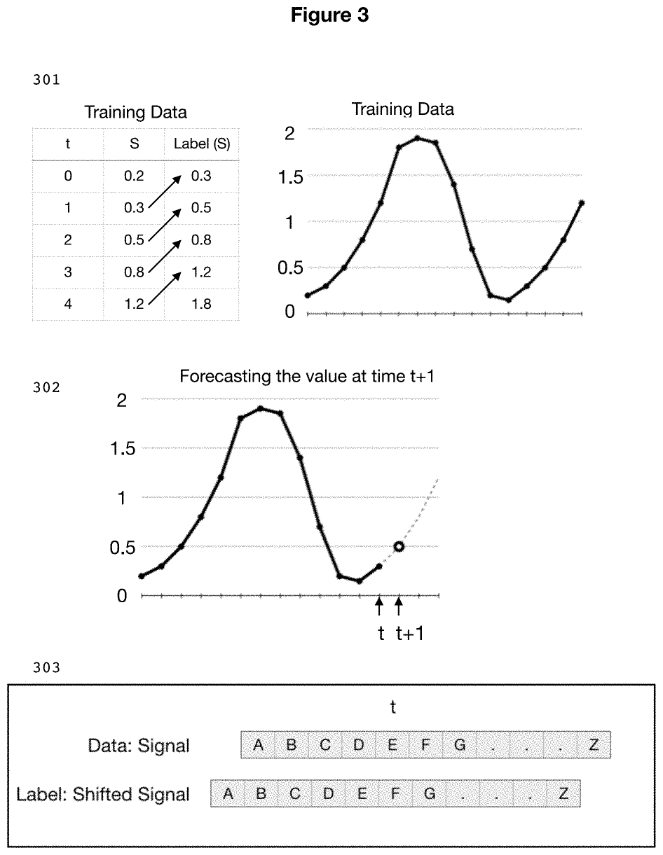

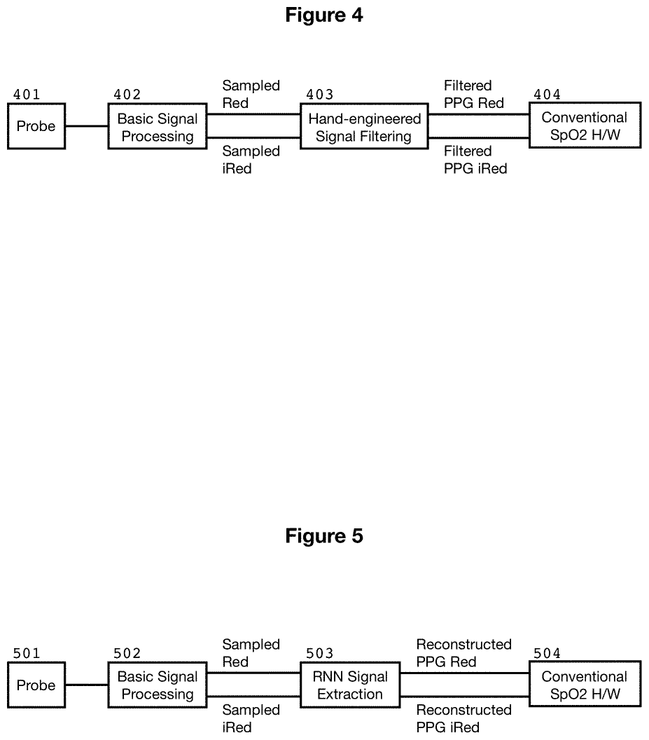

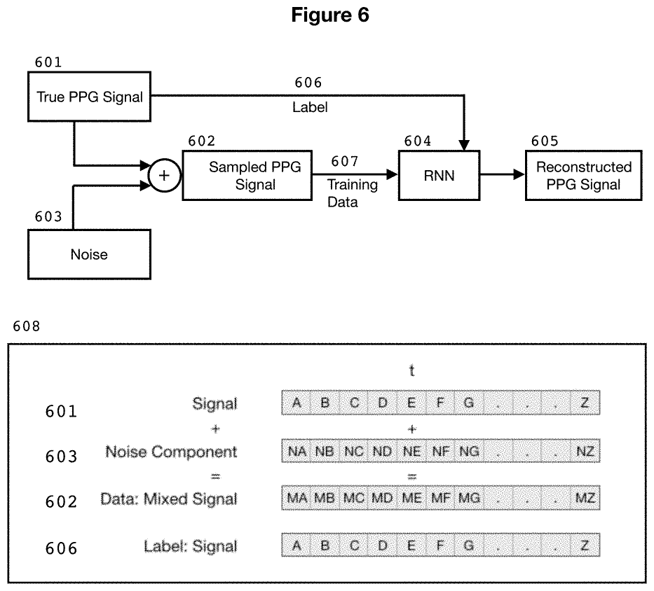

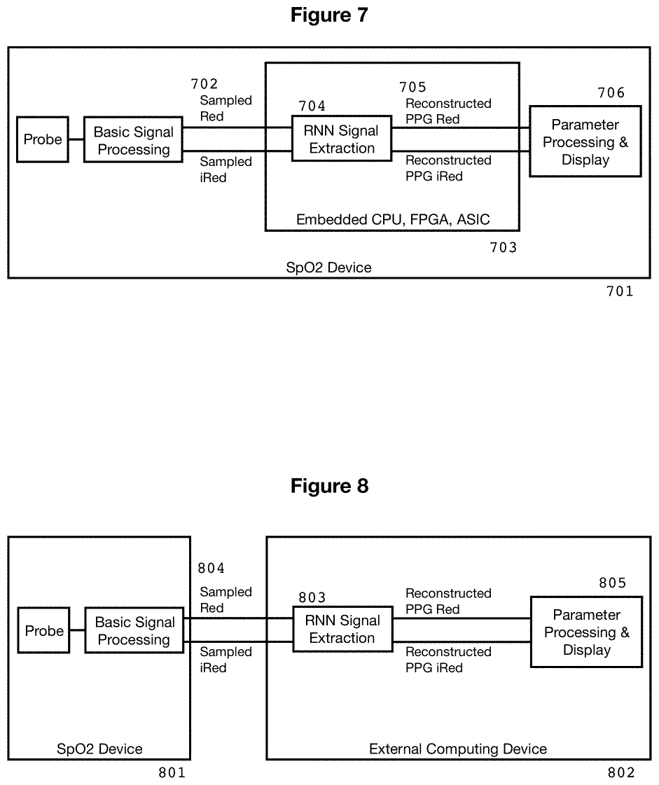

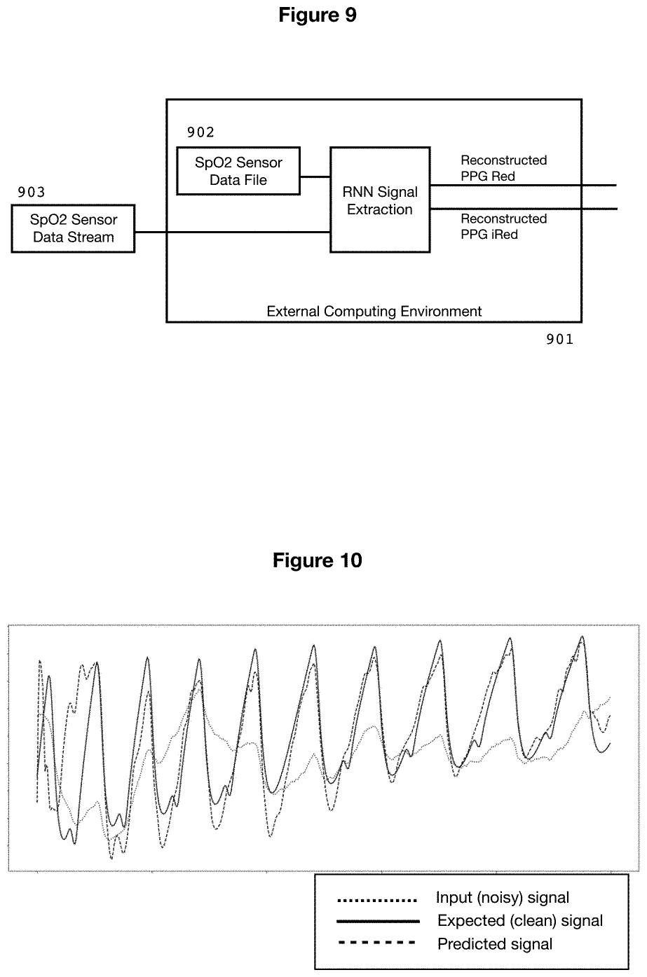

### DESCRIPTION OF EMBODIMENTS

There are several preferred embodiments possible: embodiment as part of an SpO2 sensor device, embodiment as an appendage to a sensor device (such as laptop/monitor or smartphone) and embodiment completely separate from the SpO2 sensor device (such as a hosted cloud service).

### Embodiment as Part of the SpO2 Sensor Device

The RNN signal reconstruction model can be included within the SpO2 sensor device [701] itself by instantiating the RNN model in a simple firmware/software environment within a low-cost embedded CPU. Many SpO2 devices already implement signal processing on-device and adding the RNN processing step is feasible on such a platform.

The sensor output [702] is routed to the CPU [703] and fed to the RNN software model [704]. The reconstructed signal [705] is routed from the output of the RNN model to the parameter processing and display portion [706] of the SpO2 device.

The RNN signal reconstruction model can alternatively be included within the SpO2 sensor device by instantiating the RNN model in an FPGA [702] or ASIC [702] instead of, or in addition to, the embedded CPU. This can improve real-time performance and lower the total solution cost.

### Embodiment as an Appendage to the SpO2 Sensor Device

The output from the basic SpO2 sensor device [801] can be connected to an external computing device [802], on which the RNN model [803] runs and processes the SpO2 input signal [804]. The reconstructed signal is routed to final parameter processing and display [805]. The term “external computing device” here refers to, but is not limited to, embedding in patient monitor equipment, laptops, mobile phones, tablets and any other device capable of basic computing.

Embodiment Completely Separate from the SpO2 Sensor Device

The RNN signal reconstruction can execute completely separately in time and space from the SpO2 sensor device in an external computing environment [901] such as a cloud server. An SpO2 signal reconstruction can be configured to process SpO2 samples either as a complete datafile [902] (this would be a post-processing application), or on streaming data [903] in near-realtime. The resulting reconstructed signal can then either be prepared for further immediate processing and display, or stored in a data file for later retrieval.

### Terminology

1. Photoplethysmography (PPG): a simple and low-cost optical technique that can be used to detect blood volume changes in the microvascular bed of tissue
2. Noisy data: data samples (and sequences of data samples) which consist of a mix of true physiological signal and noise components
3. Tethered computing environment: computing environment connected in near proximity to the sensor device either via physical cabling, or via wireless connectivity such as Bluetooth or WiFi.
4. Un-tethered computing environment: computing environment which is able to process signal waveforms, but which is not connected in near physical proximity of the sensor device, such as a remote server or cloud computing environment. These computing environments are sometimes referred to “off-line” computing or “batch computing” environments.
5. Waveform extraction: applying the RNN model to a noisy data signal waveform and returning a reconstruction of the original true signal waveform
6. Synthesized samples: artificially generated signal data
7. Organic samples: signal data recorded from a real-life sensor
8. Characteristic waveform: the general morphology of a signal waveform for a given type of physiological signal, including but not limited to SpO2 waveforms, ECG/EKG cardiac waveforms etc. The characteristic waveform can be quasi-periodic in nature, for example such as that of EKG generated by heartbeats.
9. Morphology: shape, in our case the general shape of a waveform when plotted as values (y) over time (x).
10. Quasi-periodic: a signal that is periodic in nature, but not exactly identical from period to period. Quasi-periodic signals have a recognizable waveform shape but may exhibit variance within that shape over time and between measurement subjects.
11. Recurrent Neural Networks (RNN): a type of neural networks which are able to recognize and construct order-dependent sequences of values.
12. Deep Learning: A class of Neural Network architectures which rely on multiple layers of neurons to learn complex and non-linear functions expressed as relationships between input data (features) and output results (labels).
13. End-to-End Deep Learning: A Deep Learning technique which bypasses the manual feature engineering phase and achieves improved neural network performance by adding more network layers and a (much) larger training set.

We claim:

1. A system of accurate reconstruction of an SpO2 signal waveform in the presence of noise comprising of:
an SpO2 sensor device which delivers red and infra-red signal components,
a Recurrent Neural Network (RNN) model trained to reconstruct a clean SpO2 waveform based on potentially noisy input signals
a method to apply forward-predicting RNN models to perform “side-casting” predictions rather than “forecasting”, thereby enabling the use of traditional end-to-end deep learning techniques,
a method to create a data set which is suitable to train the RNN to do side-casting predictions.

2. A method to create a side-cast training data set by adding noise to a clean/true SpO2 waveform sample set, and labeling the corresponding data with the clean/true SpO2 waveform sample values.

3. A method to enable side-casting by training the RNN to use time-series forecasting techniques on a data set structured for side-casting.

4. The signal acquisition system of claim 1, wherein the SpO2 waveform extraction is embodied in a sensor device as part of an embedded compute environment, including but not limited to CPU, FPGA or ASIC processing methods.

5. The signal acquisition system of claim 1, wherein the waveform extraction is embodied as an appendage to a sensor device as part of a tethered computing environment, including but not limited to laptops, embedded patient monitors and other medical devices, mobile phones and tablets.

6. The signal acquisition system of claim 1, wherein the waveform extraction is embodied in an un-tethered computing environment separately from the sensor device including but not limited to remote servers or cloud-computing environments.
